(19) United States
(12) Patent Application Publication
(10) Pub. No.: US 2007/0118189 A1
(43) Pub. Date: May 24, 2007

(54) Heart beat measuring transmitter and heart beat measuring apparatus

(76) Inventor: Hisao Nakamura, Chiba-shi (JP)

Correspondence Address: BRUCE L. ADAMS, ESQ.;SUITE 1231, 17 BATTERY PLACE, NEW YORK, NY, 10004, US

(21) Appl. No.: 11/600,558

(22) Filed: Nov. 16, 2006

(30) Foreign Application Priority Data
Nov. 24, 2005 (JP) 2005-338997

(51) Int. Cl. A61F 7/00

(52) U.S. Cl. 607/96

(57) ABSTRACT

To prevent a packing of a battery lid from being deteriorated by sweat of a user and maintain waterproof performance of an apparatus over a long period of time. A top side, that is, an upper case of a transmitter case of a heart beat measuring transmitter is provided with a battery containing portion and an openable/closable battery lid for sealing an opening of the battery containing portion in liquid tight, and an openable/closable battery lid cover for covering the battery lid. A waterproof elastic member is provided between an inner face of the battery lid cover and the upper case of the transmitter case to surround an outer side of an outer periphery of the battery lid.

### BACKGROUND OF THE INVENTION

1. Field of the Invention

The present invention relates to a heart beat measuring transmitter for transmitting by wireless from a transmitting antenna based on a cardiographic signal detected by a pair of chest electrodes attached to a chest belt, and a heart beat measuring apparatus having the heart beat measuring transmitter.

2. Description of the Prior Art

JP-A-10-155753 discloses a heart beat measuring apparatus constituted by a pair of chest electrodes attached to a chest belt, a heart beat measuring transmitter for transmitting by wireless from a transmitting antenna based on a cardiographic signal detected by the chest electrodes, and a heart beat measuring receiver for receiving a wireless signal of heart beat number data transmitted from the heart beat measuring transmitter by a receiving antenna and calculating to display a heart beat number per 1 minute by carrying out a predetermined processing by a signal processing portion.

According to the heart beat measuring transmitter of the background art, a battery containing portion is provided on a back side of a transmitter case. Further, the battery containing portion is contained with a button battery, and an opening portion thereof is sealed by ensuring a liquid tight state by an openable/closable battery lid. The liquid tight state is realized to be ensured by a waterproof packing contained at a packing containing portion provided at the opening portion of the battery containing portion.

When the heart beat measuring transmitter of the background art is mounted to the chest of a user by the chest belt, the battery lid is always brought into contact with the human body. Therefore, the battery lid is always exposed to sweat of the user and therefore, a deterioration of the waterproof packing is significantly accelerated by the sweat. Therefore, there poses a problem that a waterproof function of the heart beat measuring transmitter of the background art can be maintained only by a short period of time.

Further, according to the heart beat measuring transmitter of the background art, when the user swims while being mounted therewith, there poses a problem that a sufficient waterproof function cannot be maintained by the waterproof packing. Because the waterproof packing cannot deal with a water pressure increased in proportion to a water depth and an increase in a pressure by movement in swimming.

It is a first problem to be resolved by the invention in a heart beat measuring transmitter used by being attached to a chest belt by constituting a power source by a button battery for preventing a waterproof packing of a battery lid from being deteriorated by sweat of a user to maintain waterproof performance of the apparatus over a long period of time.

It is a second problem to be resolved by the invention in a heart beat measuring transmitter used by being attached to a chest belt by constituting a power source by a button battery to be able to ensure waterproof performance of the apparatus even in swimming.

### SUMMARY OF THE INVENTION

In order to resolve the above-described first problem, there is provided a heart beat measuring transmitter for transmitting by wireless from an antenna based on a cardiographic signal detected by a pair of chest electrodes attached to a chest belt, the heart beat measuring transmitter including a cardiographic signal detecting portion and a wireless transmitting portion and a battery containing portion for containing a battery constituting a power source thereof on a top side of a transmitter case and an openable/closable battery lid for sealing an opening of the battery containing portion in liquid tight.

In order to resolve the above-described first and second problems, in the heart beat measuring transmitter for transmitting by wireless from the antenna based on the cardiographic signal detected by the pair of chest electrodes attached to the chest belt, the heart beat measuring transmitter includes the cardiographic signal detecting portion and the wireless transmitting portion and the battery containing portion for containing the battery constituting the power source on the top side of the transmitter case and the openable/closable battery lid for sealing the opening of the battery containing portion in liquid tight, further, the openable/closable battery lid cover for covering the battery lid is provided on the top side of the transmitter case.

Further, in order to resolve the above-described first and second problems, in the heart beat measuring transmitter for transmitting by wireless from the antenna based on the cardiographic signal detected by the pair of chest electrodes attached to the chest belt, the heart beat measuring transmitter includes the cardiographic signal detecting portion and the wireless transmitting portion and the battery containing portion for containing the battery constituting the power source on the top side of the transmitter case and the openable/closable battery lid for sealing the opening of the battery containing portion in liquid tight, the openable/closable battery lid cover for covering the battery lid is provided on the top side of the transmitter case, further, the waterproof elastic member is provided between the inner face of the battery lid cover and the upper face of the transmitter case to surround the outer side of the outer periphery of the battery lid.

### BRIEF DESCRIPTION OF THE SEVERAL VIEWS OF THE DRAWINGS

A preferred form of the present invention is illustrated in the accompanying drawings in which:

FIG. 1 is a sectional view of a heart beat measuring transmitter of Embodiment 1 cut along a center line in a longitudinal direction of a case;

FIG. 2 is a plane view of the heart beat measuring transmitter of Embodiment 1 viewed from a back side of the case. However, the plane view is shown by removing a case back and almost all of electronic parts contained at inside of the case;

FIG. 3 is a plane view of the heart beat measuring transmitter of Embodiment 1 viewed from a top side of the case;

FIG. 4 is a sectional view of the heart beat measuring transmitter of Embodiment 1 cut along a center line orthogonal to the longitudinal direction of the case;

FIG. 5 is a sectional view of a heart beat measuring transmitter of Embodiment 2 cut along the center line orthogonal to the longitudinal direction of a case;

FIG. 6 is a sectional view of a heart beat measuring transmitter of Embodiment 3 cut along the center line orthogonal to the longitudinal direction of a case; and

FIG. 7 is a sectional view of the heart beat measuring transmitter of Embodiment 3 cut along the center line in the longitudinal direction of the case.

### DETAILED DESCRIPTION OF THE PREFERRED EMBODIMENTS

The invention is characterized in a heart beat measuring transmitter for transmitting by wireless from an antenna based on a cardiographic signal detected by a pair of chest electrodes attached to a chest belt, the heart beat measuring transmitter including the cardiographic signal detecting portion and a wireless transmitting portion and a battery containing portion for containing a battery constituting a power source thereof on a top side of a transmitter case and openable/closable battery lid for sealing an opening of the battery containing portion in liquid tight. Further, although a detailed description will be given of a case of using a button type battery for the battery according to an embodiment of the invention, other battery of a cylindrical type or the like will naturally do.

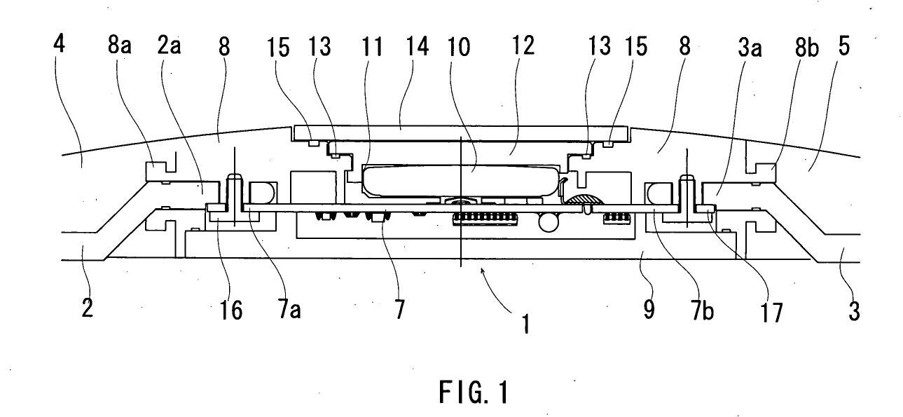

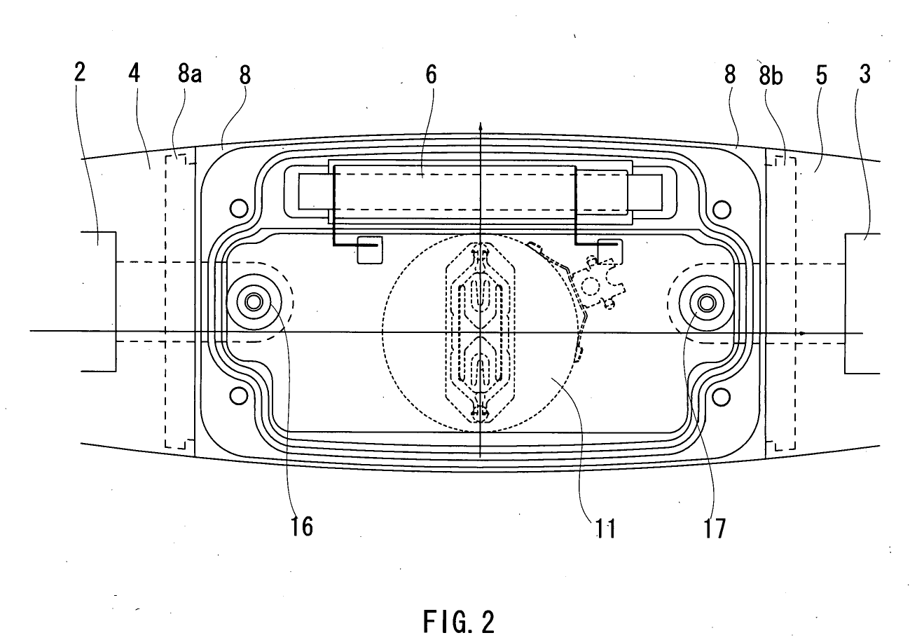

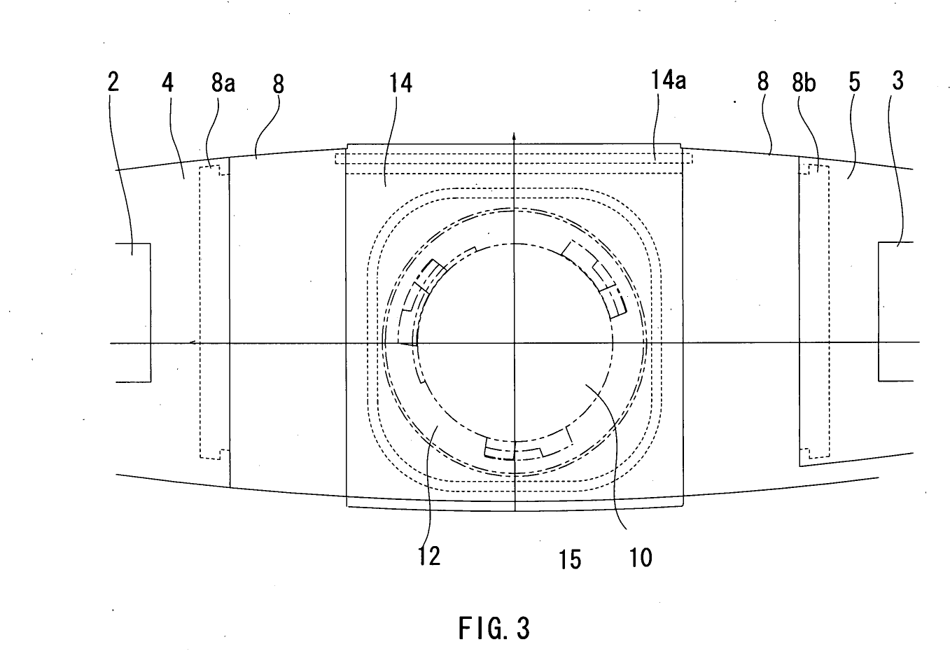

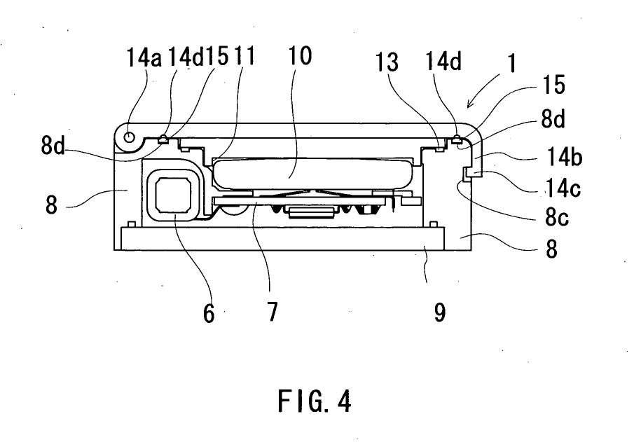

A heart beat measuring transmitter of Embodiment 1 is a heart beat measuring transmitter for transmitting by wireless from an antenna 6 based on a cardiographic signal detected by a pair of chest electrodes 2 and 3 respectively attached to chest belts 4 and 5 as shown by a sectional view of FIG. 1 cut along a center line in a longitudinal direction of a case, a plane view of FIG. 2 viewed from a back side of the case, a plane view of FIG. 3 viewed from a top side of the case, and a sectional view of FIG. 4 cut along a center line orthogonal to the longitudinal direction of the case.

The transmitter case is constituted by an upper case 8 and a lower case 9, coupled by the chest belt 4 and a coupling portion 8a and coupled by the chest belt 5 and a coupling portion 8b. An electrode 2a of the chest electrode 2 is connected to one circuit side electrode 7a of a circuit block 7 by a connecting portion 16. Further, an electrode 3a of the chest electrode 3 is connected to one circuit side electrode 7b of the circuit block 7 by a connecting portion 17.

The circuit block 7 contained in the transmitter case constitutes the cardiographic signal detecting portion and the wireless transmitting portion. A button battery 10 constituting a power source of the cardiographic signal detecting portion and the wireless transmitting portion is contained at inside of a battery containing portion 11 provided on a top side of the transmitter case, that is, a top side of the upper case 8, and an opening portion of the battery containing portion 11 is sealed by a an openable/closable battery lid 12 by way of a waterproof packing 13.

The top side of the transmitter case, that is, the top side of the upper case 8 is further attached with a battery lid cover 14 while being made to be openable/closable by a hinge 14a at one end thereof. The battery lid cover 14 is a cover having a size of completely covering the battery lid 12 in the transmitter case and occupies substantially a half of the top side of the transmitter case. As shown by FIG. 4, the battery lid cover 14 includes an engaging portion 14b vertically folded to bend at an end portion opposed to a side of the hinge 14a. Further, a front end of the engaging portion 14b is formed with an engaging projection 14c. A side face of the upper case 8 opposed to the engaging projection 14c is formed with an engaging recess portion 8c. When the battery lid cover 14 is closed, the engaging projection 14c of the battery lid cover 14 is engaged with the engaging recess portion 8c at the side face of the upper case 8 to thereby hamper the battery lid cover 14 from being opened.

A waterproof elastic member 15 is arranged between an lower face of the battery lid cover 14 and an upper face of the upper case 8 to surround an outer periphery of the battery lid. The waterproof elastic member 15 is a part of an elastic member of rubber or the like substantially in a rectangular shape constituting a center thereof the same as that of the battery lid 12 in a circular shape. The upper cover 8 is formed with an attaching groove 8d substantially in a rectangular shape for attaching the waterproof elastic member 15 to a predetermined position. Further, a lower face of the battery lid cover 14 is formed with an engaging groove 14d substantially in a rectangular shape to be engaged with the waterproof elastic member 15 attached to the attaching groove 8d. When the waterproof packing 13 of the battery lid 12 is referred to as first waterproofing means, the waterproof elastic member 15 functions as second waterproofing means.

As described above, the heart beat measuring transmitter of Embodiment 1 is provided with the cardiographic signal detecting portion and the wireless transmitting portion and the battery containing portion 11 for containing the button battery 10 constituting a power source thereof on the top side of the transmitter case and the openable/closable battery lid 12 for sealing the opening in liquid tight, the openable/closable battery lid cover 14 for covering the battery lid 12 is provided on the top side of the upper case 8, further, the waterproof elastic member 15 is provided between the inner face of the battery lid cover 14 and the upper face of the upper case 8 to surround the outer periphery of the battery lid 12.

The heart beat measuring transmitter of Embodiment 1 is constructed by the above-described constitution and therefore, always the battery lid 10 is not brought into contact with the human body, contact to a corrosion causing substance of sweat or the like is minimized and therefore, the waterproof function of the apparatus can be maintained over a long period of time. Further, by providing the battery lid cover 14, an outlook in mounting can maintain an outlook similar to that of a background art product, even when a person swims while being mounted therewith, an amount of a pressure generated by moving the human body in a pressure applied on the waterproof packing 13 of the battery lid 12 can be prevented from being directly applied on the battery lid 10 and therefore, an amount of pressure applied on the battery lid 10 is restrained, as a result, the waterproof performance can be ensured even in swimming. Furthermore, by providing the waterproof elastic member 15 functioning as the second waterproofing means, the waterproof performance of the heart beat measuring transmitter of Embodiment 1 is significantly promoted in comparison with that of the background art apparatus.

A heart beat measuring transmitter of Embodiment 2 is constituted by omitting the waterproof elastic member 15 constituting the second waterproofing means from the heart beat measuring transmitter of Embodiment 1.

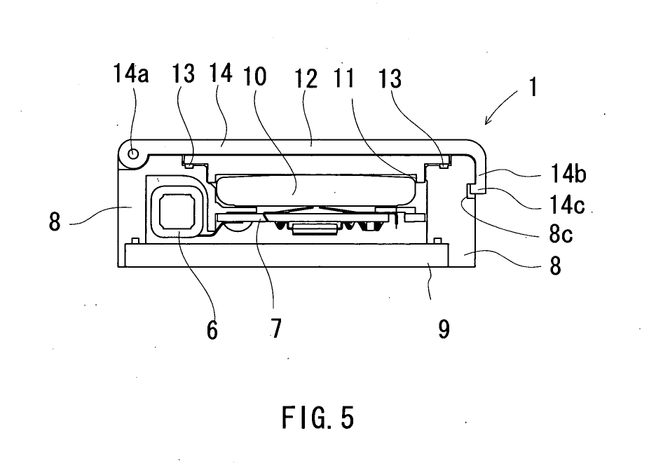

That is, as shown by a sectional view of FIG. 5 cut along the center line orthogonal to the longitudinal direction of the case, the heartbeat number calculating portion and the wireless transmitting portion and the battery containing portion 11 contained with the button battery 10 constituting the power source on the top side of the transmitter case and the openable/closable battery lid 12 for sealing the opening in liquid tight are provided, and the openable/closable battery lid cover 14 for covering the battery lid 12 is provided on the top side of the upper case 8.

The heart beat measuring transmitter of Embodiment 2 is constructed by the above-described constitution and therefore, always the battery lid 10 is not brought into contact with the human body, contact to a corrosion causing substance of sweat or the like is minimized and therefore, the waterproof function of the apparatus can be maintained over a long period of time. Further, by providing the battery lid cover 14, an outlook in mounting can maintain an outlook similar to that of the background product, even when a person swims while being mounted therewith, the amount of the pressure generated by moving the human body in the pressure applied on the waterproof packing 13 of the battery lid 12 is prevented from being applied directly on the battery lid 10 and therefore, the amount of the pressure applied on the battery lid 10 is restrained, as a result, the waterproof function can be maintained even in swimming.

A heart beat measuring transmitter of Embodiment 3 is constituted by omitting the battery lid cover 14 and the waterproof elastic member 15 from the heart beat measuring transmitter of Embodiment 1.

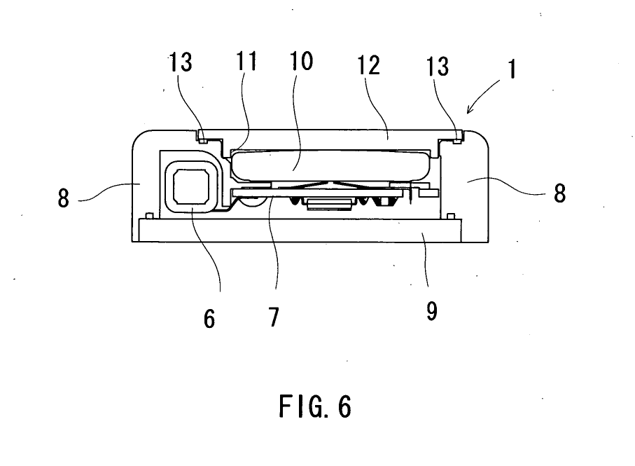

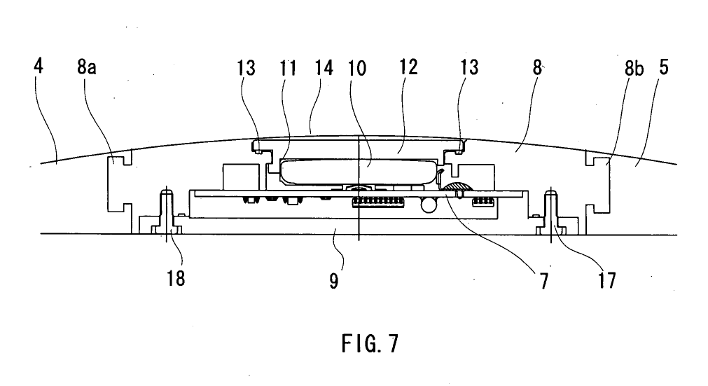

That is, the heart beat measuring transmitter of Embodiment 3 is a heart beat measuring transmitter for transmitting by wireless from the antenna 6 based on the cardiographic signal detected by the pair of chest electrodes 2 and 3 respectively attached to the chest belts 4 and 5 as shown by the sectional view of FIG. 6 cut along the center line orthogonal to the longitudinal direction of the case, and a sectional view of FIG. 7 cut along the center line in the longitudinal direction of the case.

The transmitter case is constituted by the upper case 8 and the lower case 9, coupled by the chest belt 4 and the coupling portion 8a and coupled by the chest belt 5 and the coupling portion 8b. The electrode 2a of the chest electrode 2 is connected to the one circuit side electrode 7a of the circuit block 7 by the connecting portion 16. Further, the electrode 3a of the chest electrode 3 is connected to the one circuit side electrode 7b of the circuit block 7 by the connecting portion 17.

The circuit block 7 contained at inside of the transmitter case constitutes the cardiographic signal detecting portion and the wireless transmitting portion. The button battery 10 constituting the power source of the cardiographic signal detecting portion and the wireless transmitting portion is contained at inside of the battery containing portion 11 provided on the top side of the transmitter case, that is, the top side of the upper case 8, and the opening portion of the battery containing portion 11 is sealed by the openable/closable battery lid 12 by way of the waterproof packing 13.

The heart beat measuring transmitter of Embodiment 3 is constructed by the above-described constitution and therefore, always the battery lid 10 is not brought into contact with the human body, contact to a corrosion causing substance of sweat or the like is minimized and therefore, the waterproof function of the apparatus can be maintained over a long period of time.

According to the invention, in the heart beat measuring transmitter used by being attached to the chest belt by constituting the power source by the battery, the battery lid is not directly brought into contact with the skin of a user and therefore, a packing of the battery lid is prevented from being deteriorated by sweat, and the waterproof function of the apparatus can be maintained over a long period of time. Further, a double waterproof structures can be adopted on the top side of the transmitter case and therefore, the waterproof function of the apparatus can be ensured even in swimming.

What is claimed is:

1. A heart beat measuring transmitter comprising:
a heart beat measuring transmitter for transmitting by wireless from an antenna based on a cardiographic signal detected by a pair of chest electrodes attached to a chest belt;
wherein the heart beat measuring transmitter includes a cardiographic signal detecting portion and a wireless transmitting portion and a battery containing portion for containing a battery constituting a power source thereof on a top side of a transmitter case; and
an openable/closable battery lid for sealing an opening of the battery containing portion in liquid tight.

2. A heart beat measuring transmitter according to claim 1; further comprising an openable/closable battery lid cover for covering the battery lid on the top side of the transmitter case.

3. A heart beat measuring transmitter according to claim 2, further comprising a waterproof elastic member between an inner face of the battery lid cover and an upper face of the transmitter case to surround an outer side of an outer periphery of the battery lid.

4. A heart beat measuring transmitter according to claim 2, wherein the battery lid cover is attached to the transmitter case by a hinge.

5. A heart beat measuring transmitter according to claim 2, wherein the battery lid cover is mounted openably/closably to the battery containing portion and a cylindrical recess portion for attaching the battery lid cover formed on an upper side of the battery lid.

6. A heart beat measuring apparatus including the heart beat measuring transmitter according to claim 1.

7. A heart beat measuring apparatus including the heart beat measuring transmitter according to claim 2.

8. A heart beat measuring apparatus including the heart beat measuring transmitter according to claim 3.

9. A heart beat measuring apparatus including the heart beat measuring transmitter according to claim 4.

10. A heart beat measuring apparatus including the heart beat measuring transmitter according to claim 5.
